(19) United States
(12) Patent Application Publication
(10) Pub. No.: US 2014/0379051 A1
(43) Pub. Date: Dec. 25, 2014

(54) LIGHT ACUPUNCTURE DEVICE

(71) Applicant: GCSOL TECH CO., LTD., Taichung (TW)

(72) Inventors: Jui-Fen PAI, Nantou County (TW); Tien-Jung HUANG, Taipei (TW); Fang-Chuan HO, Hsinchu (TW)

(73) Assignee: GCSOL TECH CO., LTD., Taichung (TW)

(21) Appl. No.: 13/963,106

(22) Filed: Aug. 9, 2013

(30) Foreign Application Priority Data
Jun. 19, 2013 (TW) 102211475

(51) Int. Cl. A61N 5/06

(52) U.S. Cl. CPC A61N 5/0619
USPC 607/88

(57) ABSTRACT

A light acupuncture device includes a wearing member and a stimulating module. The wearing member is adapted to be worn on a human's body, and the stimulating module is affixed to or detachably provided to wearing member. The stimulating module has a base, a light emitting unit and a driving unit. The light emitting unit is received in the base to emit specific rays. The driving unit is electrically connected to the light emitting unit to supply the light emitting unit with electricity. A user can get light acupuncture therapy while he/she wears the wearing member, and he/she can do anything he/she likes without any restriction.

The current application claims a foreign priority to the patent application of Taiwan No. 102211475 filed on Jun. 19, 2013.

### BACKGROUND OF THE INVENTION

1. Technical Field

The present invention relates to acupuncture, and more particularly to a light acupuncture device.

2. Description of Related Art

In traditional Chinese medicine (TCM), acupuncture is known around the world. The traditional acupuncture involves penetration of the skin with needles in order to stimulate certain acupuncture points on the body. However, every acupuncture point has its specific location and depth under the skin. Only a well-trained doctor can stimulate the acupuncture points right. Pain, needle phobia, and bruise are the common bad effects in acupuncture therapy.

In present days, light acupuncture therapy is invented, in which needle penetration is replaced by light. There are two types of devices for such light acupuncture therapy, fixed type and hand-held type.

The fixed type light acupuncture device has a box and a light emitter in the box to emit specific rays. The patient enters the box, and let the rays of the light emitter stimulate certain acupuncture points. It is impossible for an ordinary person to buy a fixed type light acupuncture device for acupuncture therapy at home.

The hand-held type light acupuncture device usually has a hand piece to emit specific rays. A user may hold the hand piece to let the rays stimulate certain acupuncture point. However, it is impossible for one to precisely aim the hand piece at some acupuncture points, such as the acupuncture points on the back, therefore, he/she still needs assistance in some cases.

### BRIEF SUMMARY OF THE INVENTION

In view of the above, the primary objective of the present invention is to provide a light acupuncture device, which makes one is easier to get light acupuncture therapy without any restriction.

In order to achieve the objective of the present invention, a light acupuncture device includes a wearing member, a light emitting unit and a driving unit. The wearing member is adapted to be worn on a human's body. The light emitting unit is provided on the wearing member to emit specific rays. The driving unit is electrically connected to the light emitting unit to supply the light emitting unit with electricity.

Therefore, a user can get light acupuncture therapy while he/she wears the wearing member, and he/she can do anything he/she likes without any restriction.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a perspective view of the stimulating module of a first preferred embodiment of the present invention;

FIG. 2 is a sectional view of the stimulating module of the first preferred embodiment of the present invention;

FIG. 3 is a sketch diagram of the first preferred embodiment of the present invention, showing the stimulating modules on the insoles;

FIG. 4 is a sectional view of 4-4 line in FIG. 3;

FIG. 5 is a perspective view of another stimulating module of the first preferred embodiment of the present invention;

FIG. 6 is a sectional view of a second preferred embodiment of the present invention; and

FIG. 7 is a perspective view of a third preferred embodiment of the present invention, showing the lift section in the locking slots.

### DETAILED DESCRIPTION OF THE INVENTION

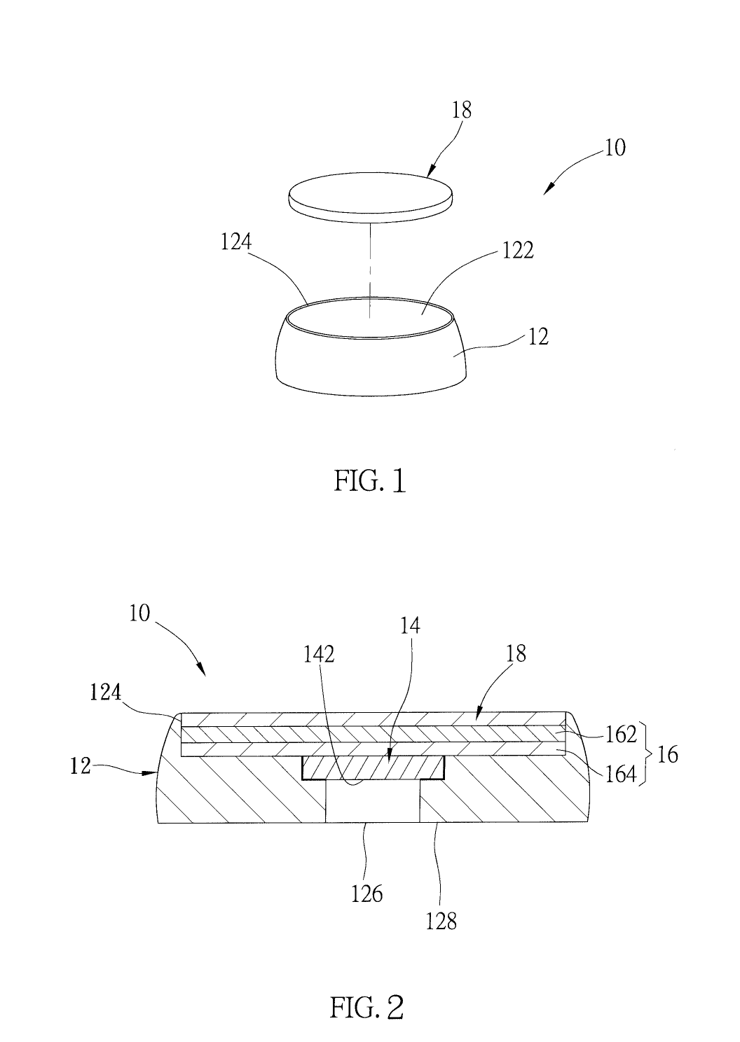

FIG. 1 and FIG. 2 show a stimulating module 10 of a light acupuncture device of the first preferred embodiment of the present invention, which includes a base 12, a light emitting unit 14, a driving unit 16 and a lid 18.

The base 12 has a room 122 therein, and a top opening 124 and a bottom opening 126 on a top and a bottom sides thereof to be communicated with the room 122. The light emitting unit 14 is received in the room 122. In an embodiment, the light emitting unit 14 is a light emitting diode (LED), which has a light emitting face 142. The light emitting face 142 is under the bottom opening 126, and is kept a distance from a bottom side 128 of the base 12. The bottom opening 126 is open on the bottom side 128. The bottom opening 126 will concentrate the rays of the light emitting diode, which is a point light source, so that the skin under the bottom opening 126 will be exposed under the rays. Besides, it may separate the light emitting unit 14 from skin when getting light acupuncture therapy to avoid a scald of skin. In another embodiment, the light emitting unit 14 is an organic light emitting diode (OLED), which is thinner and colder than LED, therefore, the distance from the bottom side 128 to the light emitting unit 14 could be shorter, or even let the OLED touch the skin.

The driving unit 16 includes a control circuit 162 and a battery 164. The driving unit 16 is received in the room 122 of the base 12 through the top opening 124. The control circuit 162 is electrically connected to the light emitting unit 14 and the battery 164 to light the light emitting unit 14 and to control the intensity of the rays of the light emitting unit 14. A switch (not shown) is connected to the control circuit 162 for manipulation to adjusting the intensity.

The lid 18 is detachably mounted in the top opening 124 of the base 12. One may open the lid 18 to repair or replace the light emitting unit 14 and the driving unit 16. The lid 18 may be plug to be squeezed into the top opening 124, or a bolt-like member to be screwed into the top opening 124.

In practice, the battery 164 of the driving unit 16 may be a button cell, a fuel cell, a Lithium battery, or other small batteries. In an embodiment, the battery 164 is a thin film battery. The thin film battery usually has integrated control circuit and battery to make the stimulating module 10 smaller. In an embodiment, the light emitting unit 14 has at least an organic light emitting diode (OLED) while the thin film battery is used. The OLED may be mounted on a substrate of the thin film battery directly to make the stimulating module 10 smaller and to integrate the elements together,

In an embodiment, the light emitting unit 14 has several light emitting devices, such as LEDs or OLEDs, to emit rays of different frequencies, or different colors.

The control circuit 162 respectively controls the intensity of the rays of each light emitting device and/or the frequencies (colors) of the rays of each light emitting device. In an embodiment, the light emitting unit 14 has several light emitting devices to emit rays of the same frequency, and the control circuit 162 respectively controls the intensity of the rays of each light emitting device only.

In an embodiment, the base 12 is transparent, and the rays of the light emitting unit 14 may emit through the base 12 without the bottom opening 126. In this embodiment, the light emitting unit 14 and the driving unit 16 are sealed in the base 12 to isolate moisture.

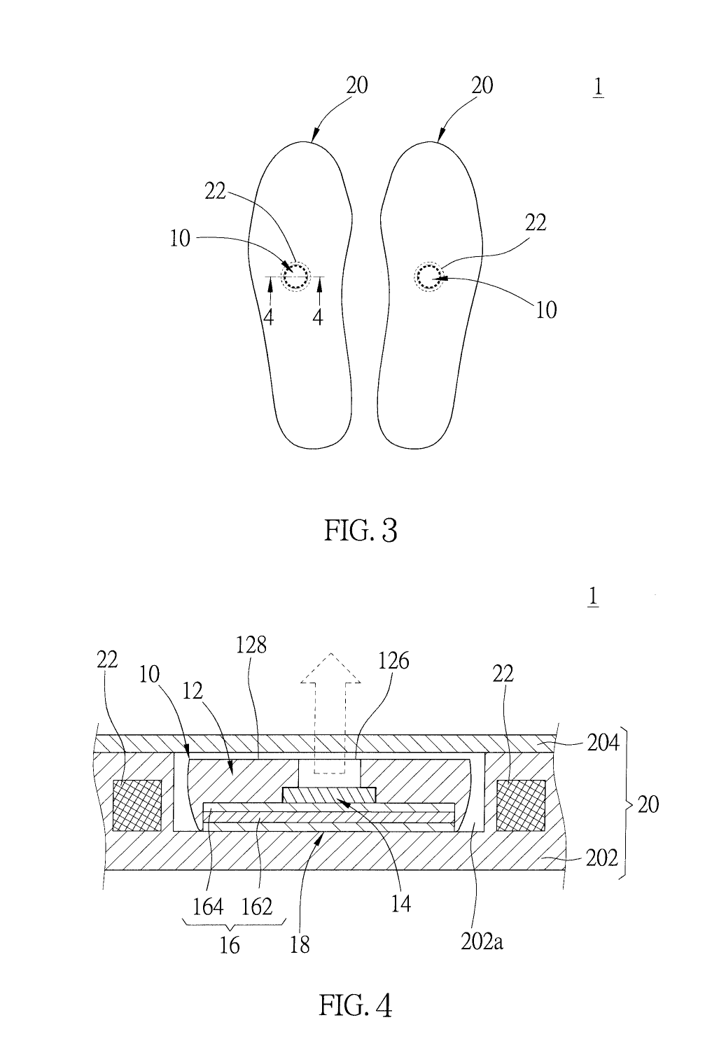

FIG. 3 and FIG. 4 show the light acupuncture device 1 of the first preferred embodiment of the present invention, which includes a wearing member and the stimulating module 10 attached to the wearing member. The wearing member is adapted to be worn on a person's body, such as clothe, paints, hat, shoes, belt, and so on, and in the second embodiment, the wearing member is a pair of insoles 20 to be put in shoes.

The insole 20 has a main body 202 and a transparent covering layer 204. The main body 202 has a recess 202a on a top side thereof to form a coupling portion of the insole 20. The stimulating module 10 is mounted in the recess 202a with the bottom opening 126 facing outside. The covering layer 204 is attached to the top side of the main member 202 and covers the recess 202a to isolate the stimulating module 10 from moisture. The rays of the light emitting unit 14 emits through the bottom opening 126 and the covering layer 204 to stimulate the acupuncture points on the feet when the user is wearing the shoes. With the light acupuncture device of the first preferred embodiment of the present invention, the user may get light acupuncture therapy when he/she wears the shoes, and he/she will look like an ordinary person and do anything he/she wants in the therapy.

The main body 202 is provided with magnetic members 22 around the recess 202a to generate a magnetic field that may enhance the effect of light acupuncture therapy.

In an embodiment, the wearing member is a pair of slippers, and the same structure may be provided on the slippers to achieve the same function.

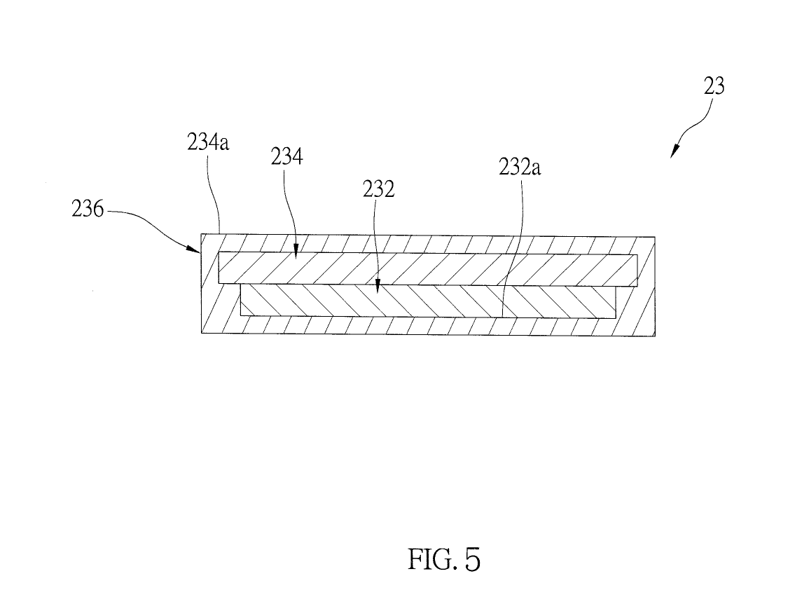

FIG. 5 shows an alternate stimulating module 23, which has a light emitting unit 232 and a driving unit 234 as well. The light emitting unit 232 is embedded in a transparent package member 236. The light emitting unit 232 has a light emitting face 232a, from which the rays emits through the package member 236. The package member 236 isolates the light emitting unit 232 from moisture. The light emitting unit 232 may be LED or OLED. The driving unit 234 may be a thin film battery, and the thin film battery covered by a protective layer 234a to isolate moisture. If the light emitting unit 232 is OLED, it may be mounted on the thin film battery directly, and the size of the stimulating module 23 will be very small, so that the stimulating module 23 is disposable.

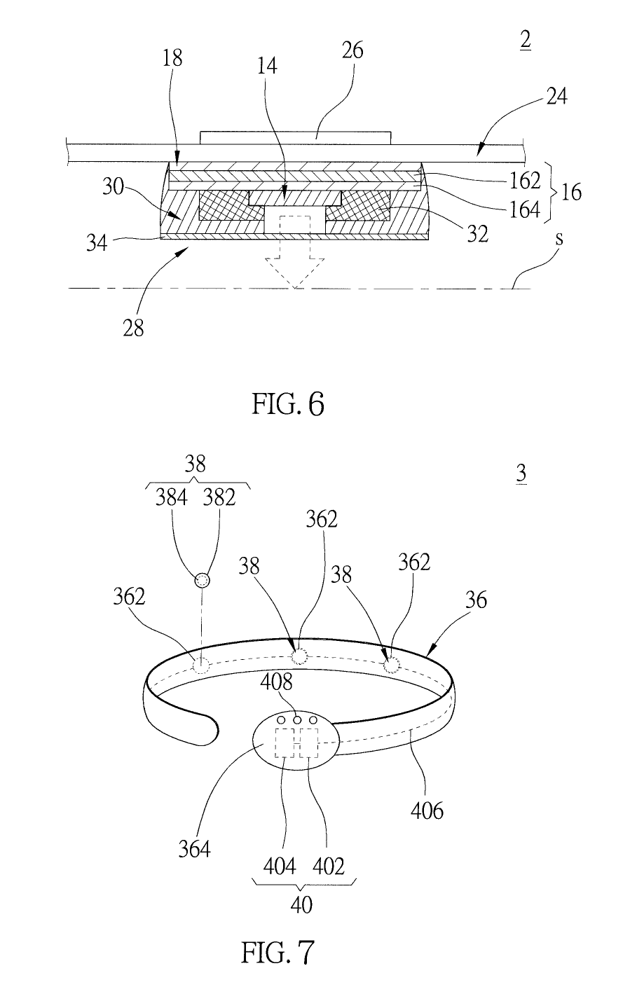

FIG. 6 shows a light acupuncture device 2 of the second preferred embodiment of the present invention, in which the wearing member is a clothe 24.

A magnetic member, which is a magnet 26, is provided to be the coupling portion of the clothe 24. A stimulating module 28, which is the same as the stimulating module 10, is provided with a plurality of magnetic members, which are magnets 32 as well, in a base 30 and around the light emitting unit 14. A transparent covering layer 34 is provided on a bottom side of the base 30 to close the bottom opening.

The stimulating module 28 is put on an interior side of the clothe 24, and the magnet 26 is put on an exterior side of the clothe 24 to attract the magnets 32 of the stimulating module 28. It may affix the stimulating module 28 to the clothe 24, and let the light emitting unit 14 emit rays on skin S to stimulate the certain acupuncture point.

The user may change the location of the stimulating module 28 on the clothe 24 easily by moving the magnet 26 because the magnet 26 is not fix to the clothe 24.

The covering layer 34 isolates the light emitting unit 14 from moisture. Because the covering layer 34 touches the skin, so the covering layer should be made of a material which has less chance to cause an allergy to skin.

FIG. 7 shows a light acupuncture device 3 of the third preferred embodiment of the present invention, in which the wearing member is a belt 36, and a plurality of stimulating modules 38 are mounted on the belt 36.

The belt 36 is provided with several recesses 362, in which the stimulating modules 38 are received. The recesses 362 are the coupling portion of the belt 36. Each stimulating module 38 has a base 382 and a light emitting unit 384 received in the base 382. In an embodiment, the base 382 is replaced by a package member to embed the light emitting unit 384 therein.

A driving unit 40 is mounted on the belt 36 to control the stimulating modules 38. The belt 36 is provided with a chamber 364, and the driving unit 40 is received in the chamber 364. The driving unit 40 has a control circuit 402 and a battery 404. The battery 404 is electrically connected to the control circuit 402 and the control circuit 402 is electrically connected to the light emitting units 384 of the stimulating modules 38 through a wire 406. The belt 36 is provided with several control keys 406, which are electrically connected to the control circuit 402 for manipulation to change the intensities, frequencies, and/or colors of the rays of the light emitting units 384. The light acupuncture device 3 of the third preferred embodiment has the same function as above.

The wearing member may be clothe, paints, shoes, hat, jewelry, or accessory to be worn on human's body to let the stimulating module stimulate certain acupuncture point(s). The user may get light acupuncture therapy anytime, anywhere by the light acupuncture device of the present invention, and he/she may do anything in the therapy.

It must be pointed out that the embodiments described above are only some preferred embodiments of the present invention. All equivalent structures which employ the concepts disclosed in this specification and the appended claims should fall within the scope of the present invention.

What is claimed is:

1. A light acupuncture device, comprising:
a wearing member, which is adapted to be worn on a human's body, having a coupling portion;
a light emitting unit engaging the coupling portion of the wearing member, wherein the light emitting unit has a light emitting face, from which specific rays emits; and
a driving unit electrically connected to the light emitting unit to supply the light emitting unit with electricity.

2. The light acupuncture device of claim 1, further comprising a base to engage the coupling portion of the wearing member, wherein the base has a room, in which the light emitting unit is received.

3. The light acupuncture device of claim 2, wherein the driving unit has a control circuit electrically connected to the light emitting unit and a battery electrically connected to the control circuit, and the control circuit is manipulated to control an intensity of the rays of the light emitting unit.

4. The light acupuncture device of claim 3, wherein the driving unit has a thin film battery.

5. The light acupuncture device of claim 3, wherein the light emitting unit has at least a light emitting diode or at least an organic light emitting diode.

6. The light acupuncture device of claim 3, wherein the light emitting unit has a plurality of light emitting devices to emit the rays of the same frequency or different frequencies.

7. The light acupuncture device of claim 6, wherein the control circuit changes intensities of the rays of the light emitting devices respectively.

8. The light acupuncture device of claim 6, wherein the light emitting devices are light emitting diodes or organic light emitting diodes.

9. The light acupuncture device of claim 2, further comprising a lid provided on the base, wherein the base has a top opening communicated with the room, and the lid closes the top opening.

10. The light acupuncture device of claim 2, wherein the driving unit is received in the room of the base.

11. The light acupuncture device of claim 2, wherein the wearing member has a chamber, in which the driving unit is received.

12. The light acupuncture device of claim 2, wherein the base is provided with a magnetic member.

13. The light acupuncture device of claim 12, further comprising a magnetic member on the wearing member to attract the magnetic member of the base, wherein the coupling portion of the wearing member is between the magnetic members.

14. The light acupuncture device of claim 1, further comprising a magnetic member provided on the wearing member.

15. The light acupuncture device of claim 2, wherein the coupling portion of the wearing member is a recess, in which the base is received.

16. The light acupuncture device of claim 15, wherein the wearing member has a main body and a transparent covering layer; the recess is provided on the main body; and the covering layer is attached to the main body and covers the recess.

17. The light acupuncture device of claim 2, further comprising a covering layer attached to the base, wherein the base has a bottom opening communicated with the room;
the rays of the light emitting unit emits through the bottom opening; and the covering layer covers the bottom opening.

18. The light acupuncture device of claim 1, wherein the driving unit has a thin film battery.

19. The light acupuncture device of claim 18, wherein the light emitting unit has at least an organic light emitting diode.

20. The light acupuncture device of claim 19, wherein the thin film battery changes an intensity of the rays of the organic light emitting diode.

21. The light acupuncture device of claim 19, further comprising a package member to embed the organic light emitting diode therein.
